(19) United States
(12) Patent Application Publication
(10) Pub. No.: US 2017/0292104 A1
(43) Pub. Date: Oct. 12, 2017

(54) MICROFLUIDIC SYSTEM AND METHOD FOR PERFUSION BIOREACTOR CELL RETENTION

(71) Applicants: MASSACHUSETTS INSTITUTE OF TECHNOLOGY, Cambridge, MA (US); NATIONAL UNIVERSITY OF SINGAPORE, Singapore (SG)

(72) Inventors: Majid EBRAHIMI WARKIANI, Singapore (SG); Jongyoon HAN, Cambridge, MA (US); Kah Ping Andy TAY, Singapore (SG); Guofeng GUAN, Singapore (SG)

(73) Assignees: MASSACHUSETTS INSTITUTE OF TECHNOLOGY, Cambridge, MA (US); NATIONAL UNIVERSITY OF SINGAPORE, Singapore (SG)

(21) Appl. No.: 15/512,003

(22) PCT Filed: Sep. 17, 2015
(86) PCT No.: PCT/US15/50604

§ 371 Date: Mar. 16, 2017

Related U.S. Application Data
(60) Provisional application No. 62/051,497, filed on Sep. 17, 2014.

(51) Int. Cl. C12M 1/00; B01L 3/00; C12M 3/06

(52) U.S. Cl. CPC C12M 29/10; C12M 23/16; B01L 3/502761; C12M 29/04; B01L 3/502753; C12M 47/04; B01L 2300/0861; B01L 2300/0681; B01L 2300/0848; B01L 2200/0647

(57) ABSTRACT

A microfluidic system for cell retention for a perfusion bioreactor is provided. The system comprises at least one inlet configured to receive a bioreaction mixture to be processed. At least one curvilinear microchannel is in fluid flow connection with the at least one inlet, the at least one curvilinear microchannel being adapted to isolate cells in the bioreaction mixture, based on cell size, along at least one portion of a cross-section of the at least one curvilinear microchannel. At least two outlets are in fluid flow connection with the at least one curvilinear microchannel. At least one outlet of the at least two outlets is configured to flow the isolated cells to be recycled to the perfusion bioreactor.

### RELATED APPLICATION

This application claims the benefit of U.S. Provisional Application No. 62/051,497, filed on Sep. 17, 2014, the entire teachings of which application are incorporated herein by reference.

### GOVERNMENT SUPPORT

This invention was made with U.S. Government support under DE-AR0000294 from ARPA-E, entitled “Scalable, Self-Powered Purification Technology for Brackish and Heavy-Metal Contaminated Water.” The U.S. Government has certain rights in the invention.

### BACKGROUND OF THE INVENTION

Mammalian cell cultures are widely used in manufacturing large and complex chemicals such as drugs and proteins for biotechnology and medicine [1]. The growing demand for these products resulted in unrelenting push for the ‘upstream’ (bioreactor operation) improvements [2]. Perfusion bioreactors have been used extensively for this purpose as they can sustain high cell number with continuous feeding of nutrients and removal of waste, as well as better control of pH and other conditions. A major challenge for continuous perfusion bioreactor design and operation is the cost and reliability of the cell retention device. A variety of techniques have been employed for cell retention or recycle, however none of these are without shortcomings [3].

Mammalian cells are useful in synthesizing large and complex chemicals such as drugs and proteins for biotechnology and medicinal purposes because they can precisely generate complex structures that the human body requires as medicine [3]. Over the past decade, mammalian cells have been employed for large-scale production of various diagnostic and therapeutic products such as monoclonal antibodies [4, 5], recombinant proteins [6] (e.g., Glycoproteins) and viral vaccines against polio [1], hepatitis B and measles. A detailed overview of the products from mammalian cells is given in elsewhere [7, 8]. Cells (yeast, algae and other cells) are also used for generating biofuels and other useful chemicals, which is increasing assuming bigger roles in the domestic energy production in the U.S. and other countries. Conventionally, cultivation of mammalian and yeast cells (for fermentation) in the large-scale can be done using various approaches such as suspension (e.g., batch, fed-batch or perfusion), roller bottles as well as micro-carriers [1, 2]. However, suspension cultivation has been used broadly in biopharmaceutical manufacturing and biofuel industry since its inception in the 1980's due to its scalability, homogeneous concentration of cells, nutrients, metabolites and product [9].

There are three types of bioreactors operating with different modes, i.e., batch, fed-batch and perfusion. These operation modes differ basically in the way nutrient supply and metabolite removal are accomplished, which can directly affect product quality, productivity and eventually cost [1]. While batch-fed process is still by far the most popular choice for biopharmaceutical production and fermentation for biofuel, recent studies shows that perfusion bioreactors will be dominant in near future. Perfusion bioreactor is ideal for manufacturing purpose as it can sustain high cell number with continual feeding of nutrients and removal of waste/product, and the parameters such as temperature and pH can be carefully tuned to maximize cell growth and ensure product batch consistency. In addition, they can produce large volumes of product from a size-limited (scalable) bioreactor on a continuous basis for extended periods of time [10], reducing capital costs. In contrast, batch and fed-batch modes are less compatible due to the lack of nutrient and waste exchange, which greatly limits productivity and necessitates large vessels. In addition, large scale centrifuge systems are needed to separate cells from product molecules post-culture, which incurs high capital cost and hard to keep sterile[11]. Especially in the second generation biofuel, organisms used are more sensitive to the product and waste-limited growth, and some of the newer biofuels (e.g., butanol) are toxic to the cells[12]. Therefore, the need for switching from batch- to perfusion-culture for these processes is expected to increase in the future.

The key parameter for successful perfusion is the retention of the majority of the cells in the bioreactor. This allows operation at relatively high flow rates with consistent product quality/stability and optimum usage of cells. Several different cell retention techniques have been used in pharmaceutical industry for separation of cells in the bioreactor during perfusion cultures. They are usually based on centrifugal action (centrifuges, hydrocyclones), filtration (cross-flow filters, hollow fibres, vortex-flow filters), gravitational/acoustic settling, ultrasonic and dielectrophoretic separation [1, 13]. Important factors that a good retention system must have are [1]:
High separation efficiency (˜100%) and high throughput (100-1000 L/day)
The device must exert minimum damage to the cells (due to shear stress) to maintain a high cell viability
The device must be stable and reliable and could be used for long-term operations (7-21 days)
The device should be cleanable, sterilizable and reusable.
Cost saving (low capital and reagent cost)

The first important category of retention devices is based on physical filtration. In this category, there are different kinds of filtration approaches such as cross-flow (or tangential) filtration, vortex-flow filters, spinfilters and hollow fibre filters. While physical filtration using microfilters has been the workhorse behind the majority of separation techniques, some major drawbacks, such as cell rupture, cell aggregation, membrane clogging and fouling exist in this mode of retention, complicate their large-scale usability [2].

Another important category, which plays a key role in pharmaceutical industry, is centrifugation. Centrifugation is a process that involves the use of the centrifugal force for the sedimentation of mixtures using a centrifuge. Despite their simplicity in usage, centrifugal devices are difficult to keep sterile and cannot be adapted for continuous-flow production [14]. It has also been reported that high acceleration intensity of 500 g can hinder cell growth up to 50% [1] and can have adverse effect on the rate of antibody production [15].

Another class of separation devices is hydrocyclone. Recently, researchers applied hydrocyclones to the separation of mammalian cells, a technique that has been previously used for yeast separation from alcoholic fermentation μ. Centrifugal forces are generated by introducing cell suspension tangentially to the cylindrical section of the device with typical pressure drop of up to 4-6 bars, with the cell experiencing ˜1000 g in the system. Due to strong swirling movement of fluid, concentrated cell suspension exits in the underflow as clarified medium exits in the overflow. Using the same separation principle as conventional centrifuges (sedimentation in a centrifugal field), hydrocyclones have many advantages for use in the biotechnology industry, such as simplicity, safety and the absence of moving parts. Researchers presented intriguing results about performance of hydrocyclones in terms of perfusion capacity but compatibility of this technique with shear-sensitive cells has to be established [1, 2]. In addition, the use of smaller cells (smaller than ˜10 μm diameter) is generally limited since current hydrocyclone systems are ineffective in capturing those smaller cells [16, 17].

Gravity settlers are probably the simplest devices which have been used in industry to retain cells. Compared to filtration, centrifugation and hydrocyclones, gravity settling is not prone to filter clogging and cell damage by high shear stresses [27]. Nonetheless, the long processing time required by gravity sedimentation is the matter of concern. In addition, the scale-up of settlers is still a problem, especially for continues processing.

Other than aforementioned techniques, there are also new methods that industry is exploring for cell retention in perfusion culture. Ultrasonic cell retention has been demonstrated but the huge vibration amplitude required in this technique causes a rise in local temperature, rendering it incompatible with heat sensitive mammalian cells and thermolabile products. Heterogeneity in temperature which causes non-uniformity in acoustic properties of the resonator also reduced productivity of this technique [1].

Dielectrophoresis method has also been recently tested for cell retention. This technique shows the disparity in separation efficiency between viable and dead cells. Nonetheless, the optimum frequency and flow rate for each type of cells have to be tuned and there has not been an industrial-scale using this technique in perfusion culture yet.

Other methods that can be used for cell retention include electrical charges and surface properties.

### SUMMARY OF THE INVENTION

In accordance with an embodiment of the invention, there is provided a microfluidic system for cell retention for a perfusion bioreactor. The system comprises at least one inlet configured to receive a bioreaction mixture to be processed; at least one curvilinear microchannel in fluid flow connection with the at least one inlet, the at least one curvilinear microchannel being adapted to isolate cells in the bioreaction mixture, based on cell size, along at least one portion of a cross-section of the at least one curvilinear microchannel; and at least two outlets in fluid flow connection with the at least one curvilinear microchannel, at least one outlet of the at least two outlets being configured to flow the isolated cells to be recycled to the perfusion bioreactor.

In further, related embodiments, the at least one curvilinear microchannel may comprise at least one spiral channel. The at least one curvilinear microchannel may comprise a plurality of curvilinear microchannels; the at least one inlet of each curvilinear microchannel of the plurality of curvilinear microchannels being in fluid flow connection with a common inlet of the microfluidic system; and the at least two outlets of each curvilinear microchannel of the plurality of curvilinear microchannels being in fluid flow connection with at least two respective common outlets of the microfluidic system. The system may comprise a plurality of channel layers attached to each other, each channel layer of the plurality of channel layers comprising at least some curvilinear microchannels of the plurality of curvilinear microchannels; the system further comprising a guide layer attached to the plurality of channel layers, the guide layer comprising the common inlet and the at least two common outlets for the plurality of curvilinear microchannels. At least one other outlet of the at least two outlets may be configured to flow at least one of: waste from the perfusion bioreactor, and a product of the perfusion bioreactor. The microfluidic system may be configured to receive a continuous flow of bioreaction mixture at the at least one inlet, and to provide a continuous flow of separated culture medium to at least one other outlet of the at least two outlets, and to provide a continuous flow of the isolated cells to be recycled to the perfusion bioreactor.

In further related embodiments, the at least one curvilinear microchannel may be adapted to isolate the cells solely due to hydrodynamic forces in the at least one curvilinear microchannel, without use of a membrane in the microfluidic system. The at least one curvilinear microchannel may have a length, and the cross-section may have a height and a width defining an aspect ratio, such that the curvilinear microchannel is adapted, by virtue of the length and the cross-section, to isolate the cells in the bioreaction mixture along the portions of the cross-section of the channel based on the cell size. The cross-section of the at least one curvilinear microchannel may be a trapezoidal cross section defined by a radially inner side, a radially outer side, a bottom side, and a top side, the trapezoidal cross section having a) the radially inner side and the radially outer side unequal in height, or b) the radially inner side equal in height to the radially outer side, and wherein the top side has at least two continuous straight sections, each unequal in width to the bottom side.

In other related embodiments, the at least one curvilinear microchannel may be adapted to filter the bioreaction mixture, such as by isolating suspended particles in the bioreaction mixture near one side of the at least one curvilinear microchannel, the suspended particles comprising the cells, and to collect clean filtrate on another side of the at least one curvilinear microchannel. The at least one curvilinear microchannel may be adapted to fractionate the bioreaction mixture, such as by isolating at least one type of smaller particles in the bioreaction mixture near an outer wall of the at least one curvilinear microchannel and isolating at least one type of larger particles in the bioreaction mixture near an inner wall of the at least one curvilinear microchannel. The at least one curvilinear microchannel may be adapted to isolate at least one of: mammalian cells and yeast cells. A product of the perfusion bioreactor may comprise at least one of: a drug, a protein, and a biofuel. A product of the perfusion bioreactor may comprise at least one of: a monoclonal antibody, a recombinant protein and a viral vaccine. The bioreaction mixture to be processed may comprise water for water pre-treatment. The bioreaction mixture may comprise a biological fluid, such as blood. The cells may comprise at least one of cancer cells, fetal cells and stem cells.

Further related methods are provided.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates steps of a fabrication process of a multilayer inertial filtration system in accordance with an embodiment of the invention, as seen in each of FIGS. 1A through 1H. In FIG. 1A, a mold design is made using SolidWorks. In FIG. 1B the mold is fabricated using conventional micromilling on the Aluminum. In FIG. 1C, soft lithography and pattern transfer to a single layer of PDMS are performed using the fabricated mold. In FIG. 1D, oxygen plasma bonding of two individual layers (i.e., manual alignment of pattern) and piercing of holes using precision punches is performed. In FIG. 1E, there is shown bonding of two individual sets of two layers after piercing holes together to make a 4-layer device. In FIG. 1F, the procedure is repeated to bond two individual sets of four layers to make an 8-layer device (i.e., this procedure can be continued to make devices with device up to 100 layers). In FIG. 1G, the 3D printed guide layer is positioned inside the device for fluid delivery. FIG. 1H shows a high-throughput device comprised of 15 layers of PDMS (60 spiral channels in total) connected to a peristaltic pump using tubing, and a 3D printed guide layer.

FIG. 2 is a schematic representation of the configuration and operational mechanism of a single spiral microfluidic chip for filtration and/or fractionation of mammalian cells with one inlet and two outlets, in accordance with an embodiment of the invention.

FIG. 3A is an optical image of a high-throughput system consisting of multiple layers of PDMS sheets with embossed microchannels (i.e., 120 spiral microchannels) bonded together for continuous cell retention from large sample volumes, in accordance with an embodiment of the invention. FIG. 3B is a sample processing workflow showing process of cell enrichment using the high throughput filtration system from spinner flasks imitating condition of a perfusion bioreactor, in accordance with an embodiment of the invention.

FIG. 4A is an exploded schematic diagram showing the assembly of multiple layers of a stack of spiral channels with a guide layer, and FIG. 4B is a schematic diagram of a single layer of the stack, in accordance with an embodiment of the invention.

FIG. 5 is a diagram showing characterization of the high-throughput microfiltration system for cell retention from a perfusion bioreactor in accordance with an embodiment of the invention. FIG. 5A shows recovery efficiency of different cell lines. FIG. 5B shows separation efficiency as a function of cell concentration. FIG. 5C shows viability and cell densities over operation time. FIG. 5D shows rate of IgG production by the Hybridoma cells over operation time.

FIG. 6 is a diagram showing FACS data obtained from a flow cytometer (Accuri C6, BD Biosciences, USA) in an experiment in accordance with an embodiment of the invention, showing the results of separation of CHO cells using a high throughput inertial filtration system at two different concentrations, mimicking condition of a perfusion bioreactor.

FIG. 7 is a diagram of phase contrast micrographs of cultures of control (unsorted) CHO cells (a-c) and sorted cells (d-f) by inertial microfiltration system in accordance with an embodiment of the invention.

FIG. 8 is a diagram of an alternative cross-section of curvilinear microchannel for use in a system according to an embodiment of the invention.

### DETAILED DESCRIPTION OF THE INVENTION

A description of example embodiments of the invention follows.

An embodiment according to the invention provides a membrane-less, clog-free microfiltration platform for ultra-high throughput (on the order of liter/min) cell separation with extremely high yield, using inertial microfluidics. A developed system in accordance with an embodiment of the invention is a highly multiplexed microfluidic device consisting of multiple layers of PDMS sheets with embossed microchannels (i.e., up to 500 spirals) bonded together for continuous size-based cell sorting from large volume of biological samples. The technique utilizes the hydrodynamic forces present in curvilinear microchannels for cell focusing and sorting.

In a system in accordance with an embodiment of the invention, cells are separated solely due to fluidic interactions driven by externally-driven flow, thus the system is inherently clog-free and can run continuously without the need for membrane filter replacement or external force fields. To characterize a system in accordance with an embodiment of the invention, while mimicking condition of a perfusion bioreactor, cell cultures were carried out using 250 mL disposable spinner flasks inside a humidified incubator for three different cell lines. Microfiltration tests were performed daily by separating the products from cells using an inertial filtration system in accordance with an embodiment of the invention inside a sterilized environment while fresh media was added to each flask after each experiment along with enriched cells. Cell densities, viability, glucose, antibody titers and pH were monitored in each sample separately. Microfiltration tests using different cell concentrations revealed usefulness of the system for continuous cell separation from bioreactors with over 95% cell separation efficiency. The viability of the sorted cells was similar to that of the unsorted (control), with more than 90% of the cells excluding the dye suggesting minimum physical damage due to the separation. Cell productivity was also assessed by measuring activity of the secreted IgG protein using an enzymatic assay. The results suggest sustainable growth of the cells and antibody production for a period of 10 days indicating the value of this new technology for separation of animal cells from the culture medium. The high throughput microfiltration system presented here can be produced with extremely low-cost using conventional micro-milling and PDMS casting. In contrast to membrane filters, this system doesn't suffer from progressive protein and cellular fouling of the filters and can be operated non-stop for a long period without any flux decline. This platform has the desirable combinations of high throughput, low cost, scalability and small foot-print, making it inherently suited for various microfiltration applications.

Microfluidics is the enabling technology for many emerging applications and disciplines, mainly in the field of biology, engineering and medicine. With the appropriate length scale that matches the scales of cells, microfluidics is well positioned to contribute significantly to cell biology [18]. Sorting cells and particles utilizing microfluidic platforms have been blooming areas of development in recent years [19]. Recently, high-throughput passive particle sorting based on inertial migration of particle inside curvilinear microchannels has been reported and has drawn wide attention as an efficient microfluidic cell separation method [20, 21]. Inertial microfluidics devices exploiting the hydrodynamic forces for particle separation rely solely on microchannel dimensions, fluidic forces and particle size to achieve separation. They have been utilized recently for various applications including cancer cell isolation, particle separation and blood fractionation [21, 22]. Due to the robust, fault-tolerant physical effects employed and high rates of operation, inertial microfluidic systems are poised to have a critical impact on high-throughput separation applications in pharmaceutical industries, environmental clean-up and physiological fluids processing [23].

In accordance with an embodiment of the present invention, there is demonstrated the usability of microfluidics for large-scale filtration applications. Table 1 gives a summary of prior methods for cell retention, discussed in the Background section above, based on five important selection criteria along with advantages and disadvantages; as compared with the microfluidic technique in accordance with an embodiment of the invention (see column labeled “Spiral System”):

TABLE 1
Existing techniques for cell retention from bioreactors (extracted from reference [1] and [2]).

| Technique/ | | | Hydro- | Gravity | Ultrasound [symbol] | Spiral |
|---|---|---|---|---|---|---|
| Criteria | Filtration | Centrifugation | cyclones | sedimentation | electrophoresis | system |
| Cell viability (%) | 50-90 | 70-85 | 80-85 | 88-100 | 70- 95 | >95 |
| Throughput | Medium | High | High | Low | Low | High |
| Running cost | High | High | Low | Low | Very High | Low |
| Scalability | Good | Good | Fair | Poor | Poor | Good |
| Separation | 63-95 | 95-100 | >85 | >85 | >95 | >95 |
| efficiency (%) | | | | | | |
| Cell | 3-30 | 3-17 | 3 | 3- [symbol] | 20-50 | 20-40 |
| Concentration | | | | | | |
| (10⁶ cells/mL) | | | | | | |
| Advantages | Applicable to | High | Low cost, | Low cost, | High | High |
| | all cell types | separation | continuous | high cell | separation | separation |
| | | efficiency | processing | viability | efficiency | efficiency, |
| | | | | | | low cost, |
| | | | | | | clog-free, |
| Disadvantages | Low | High | Not | Too slow | Low | N.A. |
| | viability, | capital cost | applicable to | | throughput | |
| | clogging | | smaller [symbol] | | High | |
| | | | | | capital cost | |

[symbol]

indicates data missing or illegible when filed

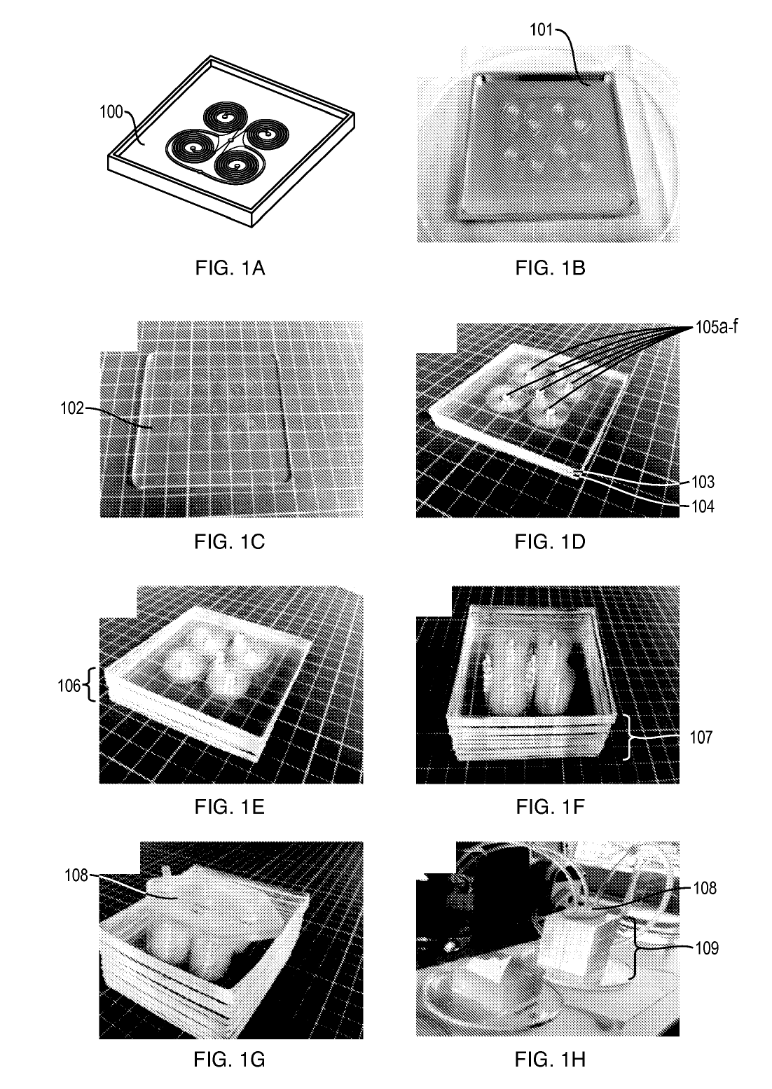

An integrated microfluidic system in accordance with an embodiment of the invention consists of multiple layer of PDMS sheets with embossed microchannels (i.e., up to 500 spiral microchannels with trapezoidal cross-section) bonded together for continuous, label/clog-free cell separation from large volume of clinical/biological samples. To simplify the operation, fluidic channels in this system are connected internally where fluid flow can be distributed through all spiral channels via a shared inlet and exit the system through collective outlets. FIG. 1 illustrates steps of a fabrication process of a multilayer inertial filtration system in accordance with an embodiment of the invention, as seen in each of FIGS. 1A through 1H. In FIG. 1A, a mold design 100 is made using SolidWorks. In FIG. 1B the mold is fabricated using conventional micromilling on the Aluminum, to produce an aluminum mold 101. In FIG. 1C, soft lithography and pattern transfer to a single layer of polydimethylsiloxane (PDMS) 102 are performed using the fabricated mold 101. In FIG. 1D, oxygen plasma bonding of two individual layers 103, 104 (i.e., manual alignment of pattern) and piercing of holes 105a-f using precision punches is performed. In FIG. 1E, there is shown bonding of two individual sets of two layers after piercing holes together to make a 4-layer device, with the four layers indicated at 106. In FIG. 1F, the procedure is repeated to bond two individual sets of four layers to make an 8-layer device, with the eight layers indicated at 107, This procedure can be continued to make devices with device up to, for example, 100 layers. In FIG. 1G, the 3D printed guide layer 108 is positioned inside the device for fluid delivery. FIG. 1H shows a high-throughput device comprised of 15 layers 109 of PDMS (60 spiral channels in total, with four spiral channels on each of the 15 layers) connected to a peristaltic pump using tubing, and a 3D printed guide layer 108.

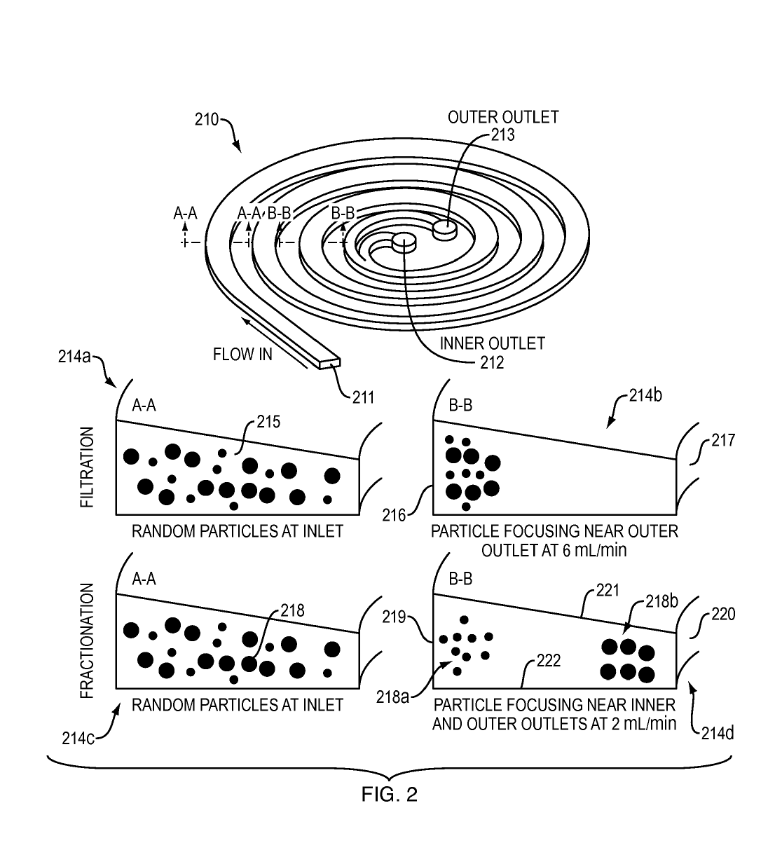

FIG. 2 is a schematic representation of the configuration and operational mechanism of a single spiral channel 210 of a microfluidic chip for filtration and/or fractionation of mammalian cells with one inlet 211 and two outlets—an inner outlet 212 and an outer outlet 213—in accordance with an embodiment of the invention.

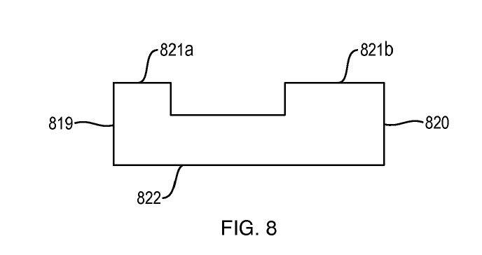

As shown schematically in FIG. 2, the system can, for example, be designed for two distinct applications, in accordance with embodiments of the invention. One is for filtration purposes, as shown in panels 214a and 214b. Here, all of the suspended particles 215 inside the fluid, which are initially randomly distributed when the fluid is flowing nearer the inlet 211 of the channel, as shown panel in 214a, can be focused near one side of the microchannel 210, i.e., normally near the outer wall 216 where there are strong vortices; and clean filtrate can be collected from another side, such as the inner wall 217 (see panel 214b). As a result of such focusing, the particles are directed to one outlet, such as the outer outlet 213, and the clean filtrate is directed to the other outlet, such as inner outlet 212. A flow rate of the fluid through the channel can be adapted to accomplish such filtration; for example, as shown in panel 214b, a flow rate of 6 mL/min was used to achieve particle focusing nearer the outer outlet 213 of the channel. Another purpose for which the system can be designed is for fractionation purposes, as shown in panels 214c and 214d. Here, suspended particles 218 are randomly distributed when the fluid is flowing nearer the inlet 211 of the channel, as shown in panel 214c; but, as a result of the flow through the spiral channel 210, as shown in panel 214d, smaller particles 218a are trapped inside Dean vortices and remain near the outer wall 219 of the channel, while bigger particles 218b are focused near the inner wall 220 of the channel, thus allowing particle separation at the inner 212 and outer 213 outlets. A flow rate of the fluid through the channel can be adapted to accomplish such fractionation; for example, as shown in panel 214d, a flow rate of 2 mL/min was used to focus particles near the inner 212 and outer 213 outlets. While FIG. 2 shows the spiral channel 210 as spiraling inwards as fluid flows from the inlet 211 to the outlets 212 and 213, it is also possible for the channel to have the inlet 211 be in the center of a spiral and have outlets 212 and 213 be at the outer edge of the channel, so that the spiral channel 210 spirals outwards as fluid flows from the inlet to the outlets. In addition, it can be seen in FIG. 2 that the channel 210 can have a trapezoidal cross-section, having a radially inner side 220, radially outer side 219, bottom side 221 and a top side 222, where the radially inner side 220 and radially outer side 219 are unequal in height. Alternatively, as shown in the embodiment of FIG. 8, the channel 810 may have a radially inner side 820 and radially outer side 819 be equal in height, while the top side has at least two continuous straight sections 821a and 821b, each unequal in width to the bottom side 822.

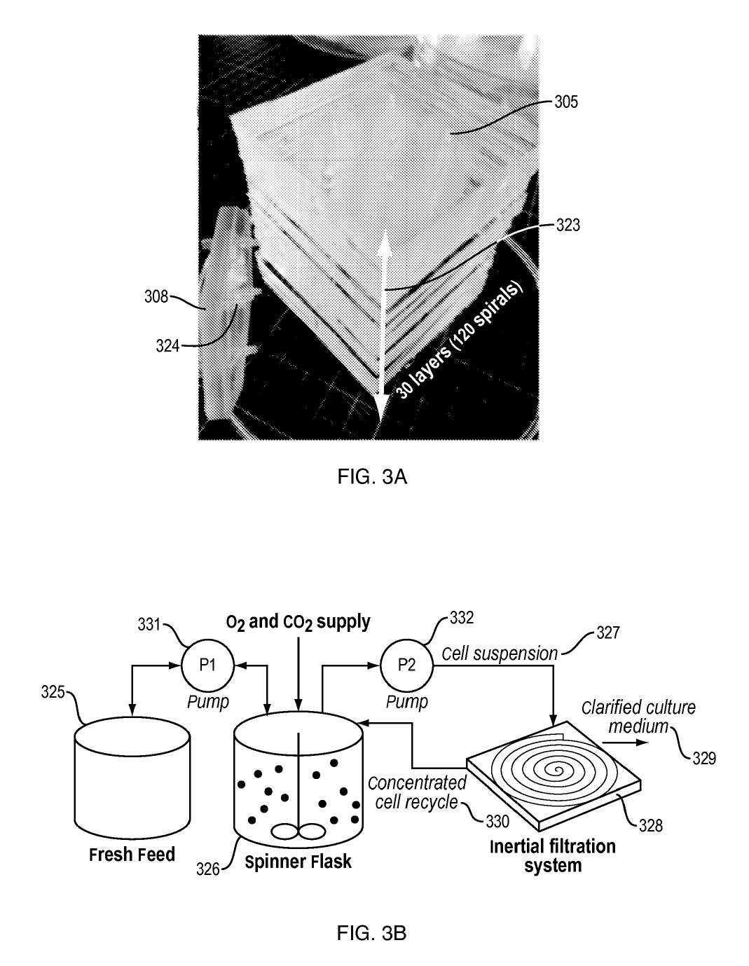

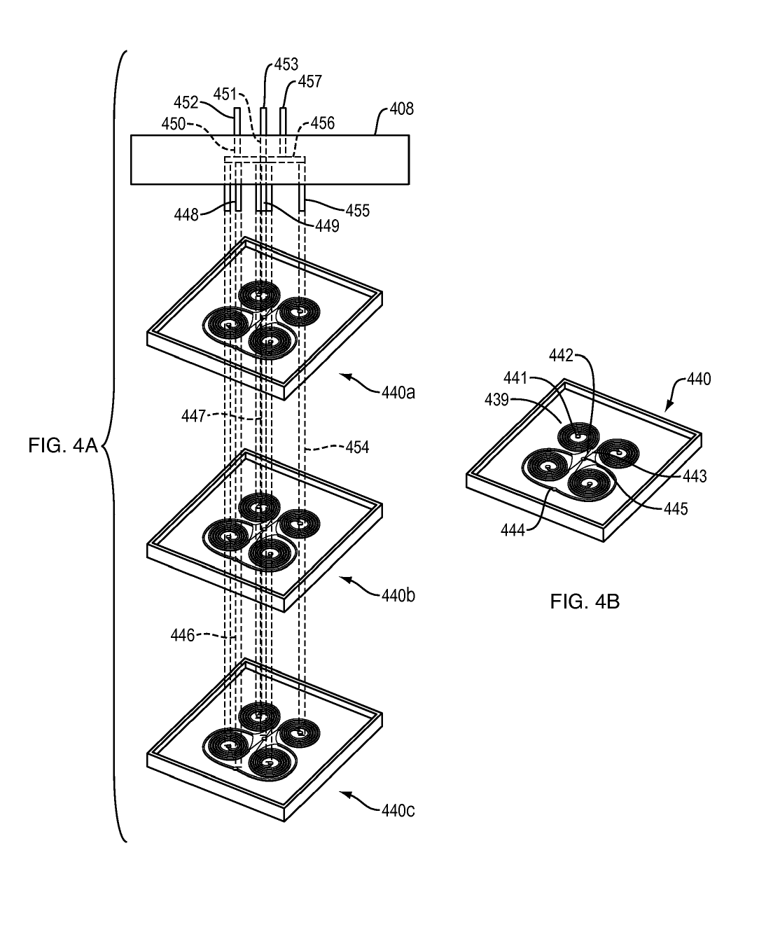

FIG. 3A is an optical image of a high-throughput system consisting of multiple layers 323 of 30 PDMS sheets with embossed microchannels (i.e., 120 spiral microchannels, where four spiral channels are on each sheet) bonded together for continuous, high throughput cell retention from large sample volumes, for a perfusion bioreactor in accordance with an embodiment of the invention. A guide layer 308 is shown beside the layers 323, in which it can be seen that inlet and outlet posts 324 extend out of the bottom side of the guide layer 308 to be inserted into holes 305 for the common inlets and outlets (discussed in FIGS. 4A and 4B, below) of the spiral channels on the multiple layers 323. In this way, the common inlets and outlets can be connected to inlet and outlet tubes that are connected to the other side of the guide layer 308. FIG. 3B is a sample processing workflow showing process of cell enrichment using the high throughput filtration system from spinner flasks imitating condition of a perfusion bioreactor, in accordance with an embodiment of the invention. Fresh feed 325 into a spinner flask 326, from which a cell suspension 327 is pumped into an inertial filtration system 328 in accordance with an embodiment of the invention. Clarified culture medium 329 is directed out of the system 328, while a concentrated cell recycle 330 is directed back to the spinner flask 326. Pumps 331 and 332 are used to flow fluid from the fresh feed 325 to the spinner flask 326 and from the spinner flask 326 to the filtration system 328. It will be appreciated that alternative flow arrangements can be used for other operations, such as fractionation, in accordance with embodiments of the invention.

FIG. 4A is an exploded schematic diagram showing the assembly of multiple layers of a stack of spiral channels with a guide layer, and FIG. 4B is a schematic diagram of a single layer of the stack, in accordance with an embodiment of the invention. In FIG. 4B, it can be seem that multiple spiral channels 439, which can be similar to the spiral channel of FIG. 2, are incorporated together on a single layer 440 of the stack. Here, four such spiral channels 439 are incorporated on the layer 440, although different numbers per layer may be used. The spiral channels 439 shown in FIG. 4B each have an inlet 441 in the center of the spiral channel, with two outlet channels 442 and 443 emerging from the outer edge of each spiral. Alternatively, the spiral channels 439 could each have an inlet on the outside of the spiral, with the two outlet channels emerging in the center of the spiral (as shown in the embodiment of FIG. 2). Within the layer 440, the outlet channels 442 and 443 from each of the spiral channels 439 on the layer are joined to two common outlet points 444 and 445 on each layer 440, which may be punched as holes through the layer. When multiple layers 440a, 440b, 440c are joined together to form a stack, as in FIG. 4A, holes through each of the common outlet points 444 and 445 (see FIG. 4B) on each stack may be joined together to form two common outlet channels 446, 447 that extend vertically through the entire stack to reach the guide layer 408. On the bottom of the guide layer, two outlet pins 448, 449 extend from the bottom of the guide layer 408 to be inserted into the top layer 440a of the stack; and are linked to outlet channels 450, 451 that extend through the guide layer 408, which connect to outlet ports 452 and 453 that extend from the top of the guide layer 408 to enable connections to tubing (not shown) that connects the system to other components of the bioreactor, for example as in the arrangement of FIG. 3B. Similarly, the inlets 441 (see FIG. 4B) may be punched as holes through each layer 440, which connect together when the stacks 440a, 440b, 440c are joined together, so that four (for example) common inlet channels 454 extend through the stack. Inlet pins 455 extend from the bottom of the guide layer 408 to be inserted into the top layer 440a of the stack. Internal inlet channels 456 of the guide layer 408 join together to flow fluid into a single inlet port 457, which can be used to connect to the external tubing (not shown), through which a fluid containing the cell suspension flows into the system. In this way, a common inlet port 457 can be used to provide inlet fluid to each inlet 441 of each of multiple spiral channels 439 on multiple different layers 440a, 440b, 440c in the system; while one common outlet port 452 receives fluid from one outlet, such as an inner outlet 212 (see FIG. 2) of a spiral channel, and another common outlet port 453 receives fluid from another outlet, such as outer outlet 213 (see FIG. 2), for each of the multiple spiral channels 439 in the system. It will be appreciated that the spiral channels of FIGS. 4A and 4B have their inlet in the center of the spiral channel, whereas those of FIG. 2 have their inlet on the outer edge of the spiral channel, but either configuration may be used.

In the embodiment of FIGS. 4A and 4B, the inlet 457 is configured to receive a bioreaction mixture to be processed. The spiral channel 439, or other curvilinear microchannel, is in fluid flow connection with the inlet 457, and is adapted to isolate cells in the bioreaction mixture, based on cell size, along at least a portion of the cross-section of the channel 439. The two outlets 452 and 453 are in fluid flow connection with the channel 439, with at least one of the outlets 452 and 453 being configured to flow isolated cells to be recycled to a perfusion bioreactor. At least one other of the outlets 452 and 453 can be configured to flow waste from the perfusion bioreactor or a product of the perfusion bioreactor. A continuous flow of bioreaction mixture can be provided to the inlet 457, while a continuous flow of separated culture medium is flowed to one of the outlets 452 and 453, and a continuous flow of isolated cells is recycled to the perfusion bioreactor. The channel 439 is adapted to isolate the cells solely due to hydrodynamic forces in the at least one curvilinear microchannel, without use of a membrane in the microfluidic system. In particular, the channel 439 has a length, and its cross-section has a height and a width defining an aspect ratio, such that the channel 439 is adapted, by virtue of its length and cross-section, to isolate the cells in the bioreaction mixture along the portions of the cross-section of the channel 439 based on the cell size, as shown, for example, in FIG. 2.

### Experimental

To evaluate the performance of a system for cell separation in accordance with an embodiment of the invention, there were employed 3 different cell lines which are widely used in industry for antibody production. These cells were cultured in suspension mode to mimic exactly bioreactor conditions. The media contains 6.3 g/L glucose and was supplemented with 8 mM L-glutamine and 100 μg/mL of an antibiotic solution. Frozen cells (CHO, MDA-MB-231 and Hybridoma) were thawed and transferred to T-25 flasks with chemically-defined medium and allowed to expand. When cultured cells reached the 90% confluency, they were filtered using a microfiltration system in accordance with an embodiment of the invention in a sterile environment and then transferred to spinner flasks for long term culture (see FIG. 3B). This procedure continued for 10 days to achieve a cell density of up to 1×10⁸, similar to the existing perfusion bioreactors.

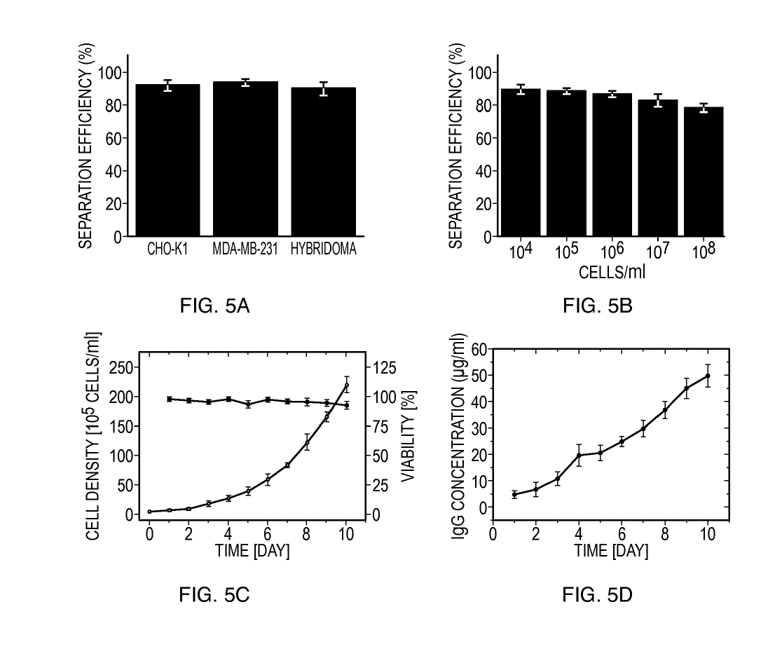

FIG. 5 is a diagram showing characterization of the high-throughput microfiltration system for cell retention from a perfusion bioreactor in accordance with an embodiment of the invention. FIG. 5A shows recovery efficiency of different cell lines. FIG. 5B shows separation efficiency as a function of cell concentration. FIG. 5C shows viability and cell densities over operation time. FIG. 5D shows rate of IgG production by the Hybridoma cells over operation time.

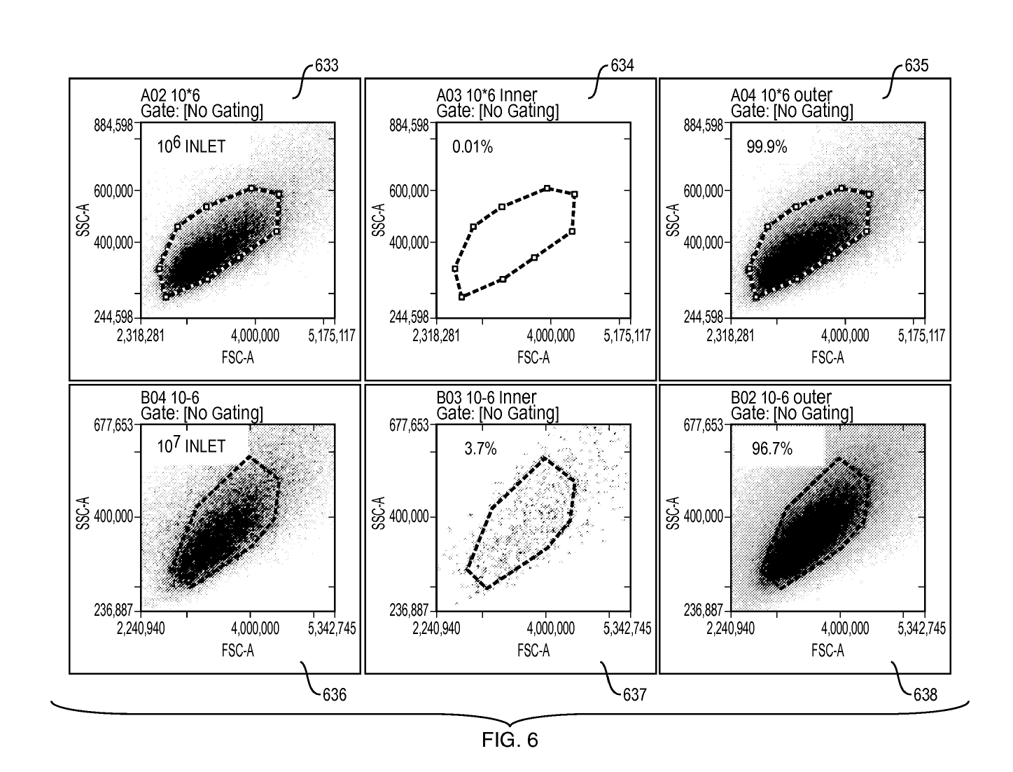

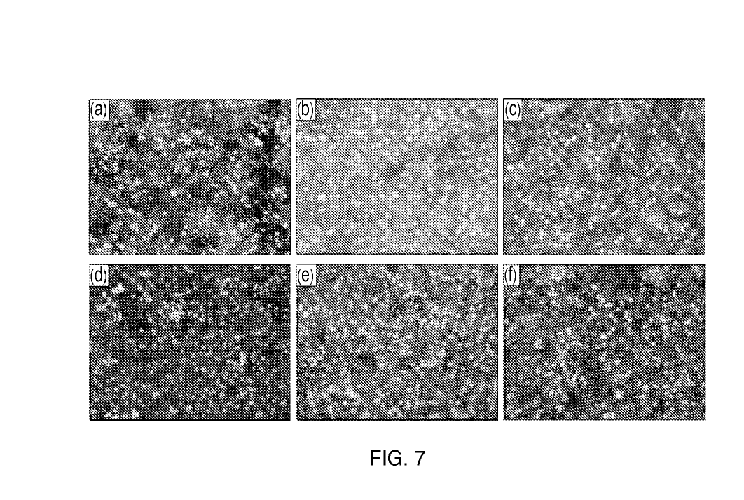

FIG. 5A depicts the separation efficiency for various cell lines, in an experiment in accordance with an embodiment of the invention. It can be seen that a high level separation (>90%) can be achieved using the system, independent of cell type. It has also been demonstrated that the system can work at high levels of cell concentration similar to those from perfusion bioreactors (see FIG. 5B and also FIG. 6), including as high as 10⁶ or greater cells/ml, such as 10⁷ and 10⁸ cells/ml. FIG. 5C shows cell growth and viability in three independent experiments, in accordance with an embodiment of the invention, where cell density (in cells/ml) is seen increasing while viability (in percent) is shown as decreasing minimally. It can be seen that a continuous cell growth can be achieved confirming minimum damage to the cells during filtration. Cell productivity (FIG. 5D) was also assessed by measuring activity of the secreted IgG protein using an enzymatic assay. The IgG protein concentration in μg/ml is seen steadily increasing over a period of 10 days. The results suggest sustainable growth of the cells and antibody production for a period of 10 days indicating the value of this new technology for separation of animal cells from the culture medium. The viability of the sorted cells was similar to that of the unsorted (control), with more than 90% of the cells excluding the dye suggesting minimum physical damage due to the separation (see FIG. 7).

FIG. 6 is a diagram showing FACS data obtained from a flow cytometer (Accuri C6, BD Biosciences, USA) in an experiment in accordance with an embodiment of the invention, showing the results of separation of CHO cells using a high throughput inertial filtration system at two different concentrations, mimicking condition of a perfusion bioreactor. In panel 633, a concentration of 10⁶ cells/ml is seen at the inlet, with the system able to produce a concentration of 0.01%, i.e., nearly full clarified of cells, at the inner outlet (see panel 634), and a concentration of 99.9% at the outer outlet (see panel 635). Similarly, in panel 636, a concentration of 10⁷ cells/ml is seen at the inlet, with a low concentration of 3.7% at the inner outlet (panel 637) and a high concentration of 96.7% at the outer outlet (panel 638).

FIG. 7 is a diagram of phase contrast micrographs of cultures of control (unsorted) CHO cells (a-c) and sorted cells (d-f) by inertial microfiltration system in accordance with an embodiment of the invention. The images indicate no significant differences between the morphology and proliferation rate of the cells suggesting high viability and sterility.

A high throughput microfiltration system in accordance with an embodiment of the invention can be produced with extremely low-cost using conventional micro-milling and PDMS casting. In contrast to membrane filters, this system doesn't suffer from progressive protein and cellular fouling of the filters and can be operated non-stop for a long period without any flux decline. This platform has the desirable combinations of high throughput, low cost, scalability and small foot-print, making it inherently suited for various microfiltration applications. In biological validation experiments, the usability of this system has been successfully shown for large-scale mammalian cell retention from bioreactors (1000 mL/min), yeast separation and stem cell fractionation. The design simplicity makes this device ideal for in-line integration with other downstream processes in perfusion bioreactors or for serving as a stand-alone, high-throughput, microfiltration/fractionation device.

A novel membrane-less microfiltration system in accordance with an embodiment of the invention is a low-cost platform for high-throughput particle separation/fractionation and can be applied in many industries where cell or particle separation is required such as breweries, pharmaceutical and water industries. As a proof of concept, there has been demonstrated the separation of animal cells from perfusion bioreactors for antibody production. This platform can be used in the water industry for water pre-treatment or can be employed in breweries/wineries for yeast removal of fermentation broth. In addition, this system has potential to be used in biomedical applications where separation of rare cells (e.g., cancer cells, fetal cells, stem cells) from a large volume of biofluids (e.g., blood) is required.

As used herein, a “curvilinear microchannel” is a microchannel in which a longitudinal axis along a direction of flow of the microchannel deviates from a straight line, and may, for example, be a spiral or sinusoidal channel.

As will be appreciated by those of ordinary skill in the art, the channel can have a variety of shapes (e.g., curved, spiral, multiloop, s-shaped, linear) provided that the dimensions of the channel are adapted to isolate cells in the bioreaction mixture, based on cell size, along at least one portion of a cross-section of the at least one curvilinear microchannel.

In one aspect, the channel is curved. In a particular aspect the channel is a spiral. The height of the spiral channel can be in a range of between about 10 μm and about 200 μm, such as about 100 μm and about 140 μm. The width of the spiral channel can be in a range of between about 100 μm and about 500 μm. The length of the spiral channel can be in a range of between about 1 cm and about 10 cm.

In one aspect, the spiral channel can be a bi-loop spiral channel. In another aspect, the spiral channel can be 2-loop spiral channel. In yet another aspect, the spiral channel can be 3-loop spiral channel. In still another aspect, the spiral channel can be 4-loop spiral channel. In another aspect, the spiral channel can be 5-loop spiral channel, etc.

The radius of the spiral channel can be adapted to yield a Dean number in a range of between about 1 and about 10, such as a radius of about 1 cm that yields a Dean number equal to about 5. The length of the spiral channel can be equal to or greater than about 3 cm, such as about 9 cm, about 10 cm, about 15 cm, and about 20 cm. The width of the spiral channel can be in a range of between about 100 μm and about 1,000 μm, such as about 200 μm, about 300 μm, about 400 μm, about 500 μm, about 600 μm, about 700 μm, about 800 μm, and about 900 μm. The height of the spiral channel can be in a range of between about 20 μm and about 200 μm, such as about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, and about 190 μm. The aspect ratio of the channel can be in a range of between about 0.1 and about 1, such as about 0.12, about 0.2, about 0.3, about 0.4, about 0.5, about 0.6, about 0.7, about 0.8, and about 0.9.

As used herein, an “aspect ratio” is the ratio of a channel's height divided by its width and provides the appropriate cross section of the channel to isolate cells in the bioreaction mixture, based on cell size, along at least one portion of a cross-section of the at least one curvilinear microchannel.

In accordance with an embodiment of the invention, microchannels, including spiral microchannels, may be used that are taught in U.S. Patent App. Pub. No. 2013/0130226 A1 of Lim et al., the entire disclosure of which is incorporated herein by reference. For example, among other things, teachings of flow rates, widths, heights, aspect ratios and lengths and other conditions relating to hydrodynamic isolation of cells may be used.

### REFERENCES

1. Castilho, L. R. and R. A. Medronho, Cell retention devices for suspended-cell perfusion cultures, in Tools and Applications of Biochemical Engineering Science 2002, Springer. p. 129-169.
2. Voisard, D., et al., Potential of cell retention techniques for large-scale high-density perfusion culture of suspended mammalian cells. Biotechnology and Bioengineering, 2003. 82(7): p. 751-765.
3. Langer, E. S., Trends in Perfusion Bioreactors. BioProcess International, 2011. 9(No. 10): p. 18-22.
4. Banik, G. G. and C. A. Heath, Partial and total cell retention in a filtration-based homogeneous perfusion reactor. Biotechnology progress, 1995. 11(5): p. 584-588.
5. Deo, Y. M., M. D. Mahadevan, and R. Fuchs, Practical Considerations in Operation and Scale-up of Spin-Filter Based Bioreactors for Monoclonal Antibody Production. Biotechnology progress, 1996. 12(1): p. 57-64.
6. Low, D., R. O'Leary, and N. S. Pujar, Future of antibody purification. Journal of Chromatography B, 2007. 848(1): p. 48-63.
7. Freshney, R. I., Culture of specific cell types2005: Wiley Online Library.
8. Pretlow, T. G. and T. P. Pretlow, Cell separation. Methods and selected applications. Vol. 21983: Academic Press, Inc.
9. Wurm, F. M., Production of recombinant protein therapeutics in cultivated mammalian cells. Nature biotechnology, 2004. 22(11): p. 1393-1398.
10. Yang, J. D., et al., Achievement of high cell density and high antibody productivity by a controlled-fed perfusion bioreactor process. Biotechnology and bioengineering, 2000. 69(1): p. 74-82.
11. Bicalho, I. C., et al., Separation of yeast from alcoholic fermentation in small hydrocyclones. Separation and Purification Technology, 2012. 87: p. 62-70.
12. Weber, C., et al., Trends and challenges in the microbial production of lignocellulosic bioalcohol fuels. Applied Microbiology and Biotechnology, 2010. 87(4): p. 1303-1315.
13. Hu, W.-S. and J. G. Aunins, Large-scale mammalian cell culture. Current opinion in biotechnology, 1997. 8(2): p. 148-153.
14. Ohashi, R., V. Singh, and J.-F. R. Hamel, Perfusion cell culture in disposable bioreactors, in Animal Cell Technology: From Target to Market2002, Springer. p. 403-409.
15. Takamatsu, H., et al., Large-scale perfusion culture process for suspended mammalian cells that uses a centrifuge with multiple settling zones. Applied microbiology and biotechnology, 1996. 45(4): p. 454-457.
16. Habibian, M., et al., Application of hydrocyclone for removal of yeasts from alcohol fermentations broth. Chemical Engineering Journal, 2008. 138(1-3): p. 30-34.
17. Yoshida, H., et al., Effect of conical length on separation performance of sub-micron particles by electrical hydro-cyclone. Powder Technology, 2012. 219(0): p. 29-36.
18. Bhagat, A. A. S., et al., Microfluidics for cell separation. Medical & biological engineering & computing, 2010. 48(10): p. 999-1014.
19. Karimi, A., S. Yazdi, and A. Ardekani, Hydrodynamic mechanisms of cell and particle trapping in microfluidics. Biomicrofluidics, 2013. 7(2): p. 021501.
20. Hou, H. W., et al., Isolation and retrieval of circulating tumor cells using centrifugal forces. Scientific Reports, 2013. 3:1259.
21. Majid, E. W., et al., Slanted spiral microfluidics for the ultra-fast, label-free isolation of circulating tumor cells. Lab Chip, 2013.
22. Bhagat, A. A. S., S. S. Kuntaegowdanahalli, and I. Papautsky, Continuous particle separation in spiral microchannels using dean flows and differential migration. Lab on a Chip, 2008. 8(11): p. 1906-1914.
23. Di Carlo, D., Inertial microfluidics. Lab on a Chip, 2009. 9(21): p. 3038-3046.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A micro fluidic system for cell retention for a perfusion bioreactor, the system comprising:
at least one inlet configured to receive a bioreaction mixture to be processed;
at least one curvilinear microchannel in fluid flow connection with the at least one inlet, the at least one curvilinear microchannel being adapted to isolate cells in the bioreaction mixture, based on cell size, along at least one portion of a cross-section of the at least one curvilinear microchannel;
wherein the cross-section of the at least one curvilinear microchannel is a trapezoidal cross section defined by a radially inner side, a radially outer side, a bottom side, and a top side, the trapezoidal cross section having the radially inner side and the radially outer side unequal in height, and wherein the top side has at least two continuous straight sections, each unequal in width to the bottom side; and
at least two outlets in fluid flow connection with the at least one curvilinear microchannel, at least one outlet of the at least two outlets being configured to flow the isolated cells to be recycled to the perfusion bioreactor.

2. The micro fluidic system of claim 1, wherein the at least one curvilinear microchannel comprises at least one spiral channel.

3. The micro fluidic system of claim 1, wherein the at least one curvilinear microchannel comprises a plurality of curvilinear microchannels;
the at least one inlet of each curvilinear microchannel of the plurality of curvilinear microchannels being in fluid flow connection with a common inlet of the micro fluidic system; and
the at least two outlets of each curvilinear microchannel of the plurality of curvilinear microchannels being in fluid flow connection with at least two respective common outlets of the microfluidic system.

4. The microfluidic system of claim 3, wherein the system comprises a plurality of channel layers attached to each other, each channel layer of the plurality of channel layers comprising at least some curvilinear microchannels of the plurality of curvilinear microchannels;
the system further comprising a guide layer attached to the plurality of channel layers, the guide layer comprising the common inlet and the at least two common outlets for the plurality of curvilinear microchannels.

5. The microfluidic system of claim 1, wherein at least one other outlet of the at least two outlets is configured to flow at least one of: waste from the perfusion bioreactor, and a product of the perfusion bioreactor.

6. The microfluidic system of claim 1, configured to receive a continuous flow of bioreaction mixture at the at least one inlet,
and to provide a continuous flow of separated culture medium to at least one other outlet of the at least two outlets,
and to provide a continuous flow of the isolated cells to be recycled to the perfusion bioreactor.

7. The microfluidic system of claim 1, wherein the at least one curvilinear microchannel is adapted to isolate the cells solely due to hydrodynamic forces in the at least one curvilinear microchannel, without use of a membrane in the microfluidic system.

8-9. (canceled)

10. The microfluidic system of claim 1, wherein the at least one curvilinear microchannel is adapted to filter the bioreaction mixture.

11. The microfluidic system of claim 10, wherein the at least one curvilinear microchannel is adapted to filter the bioreaction mixture by isolating suspended particles in the bioreaction mixture near one side of the at least one curvilinear microchannel, the suspended particles comprising the cells, and to collect clean filtrate on another side of the at least one curvilinear microchannel.

12. The microfluidic system of claim 1, wherein the at least one curvilinear microchannel is adapted to fractionate the bioreaction mixture.

13. The microfluidic system of claim 12, wherein the at least one curvilinear microchannel is adapted to fractionate the bioreaction mixture by isolating at least one type of smaller particles in the bioreaction mixture near an outer wall of the at least one curvilinear microchannel and isolating at least one type of larger particles in the bioreaction mixture near an inner wall of the at least one curvilinear microchannel.

14. The microfluidic system of claim 1, wherein the at least one curvilinear microchannel is adapted to isolate at least one of: mammalian cells and yeast cells.

15. The microfluidic system of claim 1, wherein a product of the perfusion bioreactor comprises at least one of: a drug, a protein, and a biofuel.

16. The microfluidic system of claim 1, wherein a product of the perfusion bioreactor comprises at least one of: a monoclonal antibody, a recombinant protein and a viral vaccine.

17. The microfluidic system of claim 1, wherein the bioreaction mixture to be processed comprises water for water pre-treatment.

18. The micro fluidic system of claim 1, wherein the bioreaction mixture comprises a biological fluid.

19. The micro fluidic system of claim 1, wherein the bioreaction mixture comprises blood.

20. The micro fluidic system of claim 1, wherein the cells comprise at least one of cancer cells, fetal cells and stem cells.

21. A method for cell retention for a perfusion bioreactor, the method comprising:
flowing a bioreaction mixture to be processed through at least one inlet of a microfluidic cell retention system of the perfusion bioreactor;
flowing the bioreaction mixture from the at least one inlet through at least one curvilinear microchannel of the cell retention system in fluid flow connection with the at least one inlet, thereby isolating cells in the bioreaction mixture, based on cell size, along at least one portion of a cross-section of the at least one curvilinear microchannel; and
flowing the isolated cells to be recycled to the perfusion bioreactor through at least one outlet of at least two outlets of the cell retention system that are in fluid flow connection with the at least one curvilinear microchannel.

22-40. (canceled)
